(19) United States
(12) Patent Application Publication
(10) Pub. No.: US 2026/0022374 A1
(43) Pub. Date: Jan. 22, 2026

(54) Use Of Serine/Arginine-Rich Splicing Factor 1 (SRSF1) As Therapeutic Target For Aortic Dissection

(71) Applicant: Beijing Friendship Hospital, Capital Medical University, Beijing (CN)

(72) Inventors: Chunmei CAO, Beijing (CN); Weiwei AN, Beijing (CN); Yang LI, Beijing (CN); Xun WANG, Beijing (CN); Rilei DAI, Beijing (CN)

(21) Appl. No.: 18/825,116

(22) Filed: Sep. 5, 2024

(30) Foreign Application Priority Data
Jul. 19, 2024 (CN) 202410977311.7

(51) Int. Cl. C12N 15/113; A61K 45/06; A61P 9/14; G01N 33/50; G01N 33/68

(52) U.S. Cl. CPC C12N 15/113; A61K 45/06; A61P 9/14; G01N 33/5023; G01N 33/6893; C12N 2310/11; C12N 2310/531; G01N 2333/4703

(57) ABSTRACT

Use of a serine/arginine-rich splicing factor 1 (SRSF1) as a therapeutic target for aortic dissection is provided, which belonging to the technical field of biomedicine. The SRSF1 can be used as a target to screen a drug for preventing and/or treating aortic dissection. SRSF1 expression is upregulated in both β-aminopropionitrile (BAPN)-induced aortic dissection model mice and aortic dissection patients. Mice with SRSF1 knockdown in vascular smooth muscle cells (VSMCs) are less susceptible to aortic dissection and elastic fiber degradation induced by BAPN. Mice with high expression of SRSF1 in VSMCs are more susceptible to aortic dissection and elastic fiber degradation induced by BAPN. Mechanistically, the SRSF1 can regulate the inflammatory phenotype of vascular smooth muscle and promote the degradation of vascular extracellular matrix. Knocking down the SRSF1 may improve the above phenomenon, thereby providing a new target for the treatment of aortic dissection.

### CROSS REFERENCE TO RELATED APPLICATION

This patent application claims the benefit and priority of Chinese Patent Application No. 202410977311.7 filed with the China National Intellectual Property Administration on Jul. 19, 2024, the disclosure of which is incorporated by reference herein in its entirety as part of the present application.

### REFERENCE TO SEQUENCE LISTING

A computer readable XML file entitled “GWP20240403110_seqlist”, that was created on Jul. 27, 2024, with a file size of about 9606 bytes, contains the sequence listing for this application, has been filed with this application, and is hereby incorporated by reference in its entirety.

### TECHNICAL FIELD

The present disclosure belongs to the technical field of biomedicine, and in particular to use of a serine/arginine-rich splicing factor 1 (SRSF1) as a therapeutic target for aortic dissection.

### BACKGROUND

Aortic dissection (AD) is a life-threatening cardiovascular disease caused by separation (dissection) of the aortic wall due to tearing in the aortic intima or bleeding within the aortic wall. Aortic dissection develops suddenly, progresses rapidly, and can be fatal. If not promptly diagnosed and left treated, this disease may lead to the death of patients in a short period of time. Alternatively, due to blood entering the tunica media, true and false cavities are formed, leading to ischemia and hypoxia of the distal major organs. Complications such as stroke caused by cerebral ischemia, acute renal failure caused by renal artery involvement or renal ischemia, and intestinal necrosis and stress ulcer/bleeding caused by gastrointestinal ischemia can occur, with an extremely high mortality rate or disability rate. However, the pathogenesis still remains unclear for this highly lethal and destructive vascular disease. At present, except for surgical intervention, there is no effective drug to prevent the occurrence of aortic dissection and block the rupture of aortic dissection. Moreover, surgical treatment is difficult, expensive, and associated with high mortality and disability rates. Therefore, active research into the causes and pathological mechanism of aortic dissection, as well as identification of therapeutic targets, is crucial for effectively preventing and treating this disease.

### SUMMARY

In view of this, an objective of the present disclosure is to provide use of an SRSF1 as a target in screening a drug for prevention and/or treatment of aortic dissection. In the present disclosure, a selected drug can effectively prevent and/or treat the aortic dissection, thus providing a new target for treating the aortic dissection.

Another objective of the present disclosure is to provide use of an SRSF1 inhibitor in preparation of a drug for prevention and/or treatment of aortic dissection.

Another objective of the present disclosure is to provide a drug for preventing and/or treating aortic dissection.

To achieve the above objective, the present disclosure provides the following technical solutions.

The present disclosure provides use of an SRSF1 as a target in screening a drug for prevention and/or treatment of aortic dissection.

In some embodiments, the SRSF1 is selected from the group consisting of an SRSF1 gene and a SRSF1 protein.

In some embodiments, overexpression of the SRSF1 promotes occurrence of the aortic dissection, and overheating low expression of the SRSF1 inhibits the occurrence of the aortic dissection.

The present disclosure further provides use of an SRSF1 inhibitor in preparation of a drug for prevention and/or treatment of aortic dissection.

In some embodiments, the inhibitor is selected from the group consisting of an SRSF1 gene inhibitor and an SRSF1 protein inhibitor.

In some embodiments, the SRSF1 gene inhibitor is selected from the group consisting of a nucleic acid molecule, a nucleic acid construct, and a lentivirus.

In some embodiments, the nucleic acid molecule is a short hairpin RNA (shRNA), and the shRNA includes a sense strand fragment, an antisense strand fragment, and a stem-loop structure ligating the sense strand fragment and the antisense strand fragment; sequences of the sense strand fragment and the antisense strand fragment are complementary; and the sequence of the sense strand fragment is any one selected from the group consisting of SEQ ID NO: 1, SEQ ID NO: 2, and a sequence complementary thereto.

In some embodiments, the SRSF1 protein inhibitor is selected from the group consisting of an SRSF1 protein antibody, an SRSF1 protein binding molecule, and an SRSF1 protein degradation agent.

The present disclosure further provides a drug for preventing and/or treating aortic dissection, including a pharmaceutically acceptable carrier and an SRSF1 inhibitor.

In some embodiments, the inhibitor is selected from the group consisting of an SRSF1 gene inhibitor and an SRSF1 protein inhibitor.

The embodiments of the present disclosure have the following beneficial effects:

In the present disclosure, it is found that SRSF1 expression is upregulated in both β-aminopropionitrile (BAPN)-induced aortic dissection model mice and aortic dissection patients. Mice with SRSF1 knockdown in vascular smooth muscle cells (VSMCs) are less susceptible to aortic dissection and elastic fiber degradation induced by BAPN. Mice with high expression of SRSF1 in VSMCs are more susceptible to aortic dissection and elastic fiber degradation induced by BAPN. Mechanistically, SRSF1 can regulate the inflammatory phenotype of vascular smooth muscle and promote the degradation of vascular extracellular matrix. Knocking down the SRSF1 may improve the above phenomenon. SRSF1 is used as a target in screening a drug for prevention and/or treatment of aortic dissection, such that a selected drug can effectively prevent and/or treat the aortic dissection, thus providing a new target for treating the aortic dissection.

### BRIEF DESCRIPTION OF THE DRAWINGS

FIGS. 1A-1H show expression of SRSF1 in patients with aortic dissection and mice; where FIG. 1A: mRNA expression of SRSF1 in vascular tissues of patients with aortic dissection and healthy people; FIG. 1B: Western blot analysis and quantification of SRSF1 protein in vascular tissues of patients with aortic dissection and healthy people; FIG. 1C: immunofluorescence staining and quantification of SRSF1 protein in vascular tissues of patients with aortic dissection and healthy people; FIG. 1D: Western blot analysis and quantification of SRSF1 protein in vascular tissues of mouse aortic dissection; FIG. 1E: mRNA expression of SRSF1 in vascular tissues of mouse aortic dissection; FIG. 1F: Immunohistochemical (IHC) staining and quantification of SRSF1 in vascular tissues of mouse aortic dissection; FIG. 1G: immunofluorescence staining of SRSF1 in vascular tissues of mouse aortic dissection; FIG. 1H: quantification of immunofluorescence staining of SRSF1 in vascular tissues of mouse aortic dissection; NOTE: Healthy represents healthy people, Dissection represents patients with aortic dissection, and D7, D14, D21, and D28 represent the 7th, 14th, 21st, and 28th days of BAPN induction, respectively;

FIGS. 2A-2G show influence of knocking down SRSF1 on the occurrence of aortic dissection; where FIG. 2A: overall vascular image of aortic dissection in BAPN-induced control and knockout mice; FIG. 2B: mouse survival rate; FIG. 2C: aortic dissection formation ratio in BAPN-induced control and knockout mice; FIG. 2D: ratio of different degrees of aortic dissection in overall blood vessels in BAPN-induced control and knockout mice; FIG. 2E: ratio of different degrees of aortic dissection in ascending aorta, descending aorta, aortic arch, and abdominal aorta in BAPN-induced control and knockout mice; FIG. 2F: elastin staining; FIG. 2G: quantification of the elastin staining;

FIGS. 3A-3F show influence of knocking down SRSF1 on inflammation level and extracellular matrix degradation; where FIG. 3A: mRNA expression of inflammatory factors in control and knockout mice; FIG. 3B: Western blot analysis of inflammatory factors in control and knockout mice; FIG. 3C: quantification of protein expression of inflammatory factors in control and knockout mice; FIG. 3D: mRNA expression of matrix metalloproteinases in control and knockout mice; FIG. 3E: Western blot analysis of matrix metalloproteinases in control and knockout mice; FIG. 3F: quantification of protein expression of matrix metalloproteinases in control and knockout mice;

FIGS. 4A-4H show influence of overexpression of SRSF1 on the occurrence of aortic dissection; where FIG. 4A: overall vascular image of aortic dissection in BAPN-induced control and overexpression mice; FIG. 4B: mouse survival rate; FIG. 4C: aortic dissection formation ratio in BAPN-induced control and overexpression mice; FIG. 4D: ratio of aortic dissection of different degrees in the overall blood vessels in BAPN-induced control and overexpression mice; FIG. 4E: ratio of aortic dissection of different degrees in ascending aorta, descending aorta, aortic arch, and abdominal aorta in BAPN-induced control and overexpression mice; FIG. 4F: elastin staining and quantification; FIG. 4G: immunofluorescence staining and quantification of inflammatory factors in control and knockout mice; FIG. 4H: immunofluorescence staining and quantification of matrix metalloproteinases in control and knockout mice;

FIGS. 5A-5I show that SRSF1 promotes aortic dissection by upregulating FosB; where FIG. 5A: heat map of vascular RNA-seq results of control mice and knockout mice with or without BAPN stimulation; FIG. 5B: the intersection of upregulated or downregulated genes in control mice under BAPN treatment compared with water feeding, and upregulated or downregulated genes in knockout mice and control mice under BAPN treatment; FIG. 5C: GO enrichment results of upregulated genes in control mice under BAPN treatment compared with water feeding, and downregulated genes in knockout mice and control mice under BAPN treatment; FIG. 5D: heat map of inflammatory factors expression in control mice and knockout mice; FIG. 5E: heat map of extracellular matrix expression in control mice and knockout mice; FIG. 5F: heat map of matrix metalloproteinase expression in control mice and knockout mice; FIG. 5G: schematic diagram of SRSF1 regulated gene screening; FIG. 5H: mRNA expression of API family genes in control mice and knockout mice; FIG. 5I: Western blot analysis and quantification of FosB in control mice and knockout mice;

FIGS. 6A-6E show that SRSF1 promotes increased inflammatory levels in cells by upregulating FosB; where FIG. 6A: mRNA expression of FosB and matrix metalloproteinases after SRSF1 knockout; FIG. 6B: Western blot analysis and quantification of FosB and matrix metalloproteinases after SRSF1 knockout; FIG. 6C: mRNA expression of FosB and matrix metalloproteinases after SRSF1 overexpression; FIG. 6D: Western blot analysis and quantification of FosB and matrix metalloproteinases after SRSF1 overexpression; FIG. 6E: influence of overexpressing SRSF1 on the expression of FosB and matrix metalloproteinases after FosB knckout; and

FIGS. 7A-7F show influence of SRSF1 inhibitor on aortic dissection; where FIG. 7A: diagram of Labetalol and SRSF1 binding pattern; FIG. 7B: Labetalol inhibiting the binding of SRSF1 and downstream RNA; FIG. 7C: overall image of blood vessels in aortic dissection after inhibitor treatment of mice; FIG. 7D: mouse mortality rate; FIG. 7E: image showing elastin staining of mouse blood vessel cross section and quantification; FIG. 7F: immunofluorescence staining and quantification of inflammatory factors and matrix metalloproteinases in aortic dissection blood vessels of mice after inhibitor treatment.

### DETAILED DESCRIPTION OF THE EMBODIMENTS

The present disclosure provides use of an SRSF1 as a target in screening a drug for prevention and/or treatment of aortic dissection.

In the present disclosure, the SRSF1 is selected from the group consisting of an SRSF1 gene and a SRSF1 protein. As an example, the human SRSF1 gene has a sequence number: Gene ID: 6426; the mouse SRSF1 gene has a sequence number: Gene ID: 110809. SRSF1 gene or protein expression is upregulated in both β-aminopropionitrile (BAPN)-induced aortic dissection model mice and aortic dissection patients. Mice with SRSF1 knockdown in VSMCs are less susceptible to aortic dissection and elastic fiber degradation induced by BAPN. Mice with high expression of SRSF1 in VSMCs are more susceptible to aortic dissection and elastic fiber degradation induced by BAPN. Mechanistically, SRSF1 can regulate the inflammatory phenotype of vascular smooth muscle and promote the degradation of vascular extracellular matrix. Knocking down the SRSF1 may improve the above phenomenon, thereby providing a new target for the treatment of aortic dissection.

In the present disclosure, as an embodiment, influence of the drug on the expression of SRSF1 is detected to screen the drug that can prevent and/or treat aortic dissection; if the drug can reduce the expression of SRSF1 gene or protein, it is considered to haven the effect of preventing and/or treating aortic dissection.

The present disclosure further provides use of an SRSF1 inhibitor in preparation of a drug for prevention and/or treatment of aortic dissection.

In the present disclosure, the inhibitor is selected from the group consisting of an SRSF1 gene inhibitor and an SRSF1 protein inhibitor. The SRSF1 gene inhibitor is selected from the group consisting of a nucleic acid molecule, a nucleic acid construct, and a lentivirus. The nucleic acid molecule includes SRSF1 gene-specific siRNA, SRSF1 gene-specific shRNA, SRSF1 gene-specific microRNA, and nucleic acid molecules that inhibit SRSF1 gene promoters. As an embodiment, the nucleic acid molecule is a short hairpin RNA (shRNA), and the shRNA includes a sense strand fragment, an antisense strand fragment, and a stem-loop structure (loop sequence) ligating the sense strand fragment and the antisense strand fragment; sequences of the sense strand fragment and the antisense strand fragment are complementary; and the sequence of the sense strand fragment is any one selected from the group consisting of SEQ ID NO: 1, SEQ ID NO: 2, and a sequence complementary thereto. There is no particular limitation on the sequence of the stem-loop structure, which can be routinely selected according to actual demands. The nucleic acid construct carries a gene fragment encoding the nucleic acid molecule and can express the nucleic acid molecule. The lentivirus is formed by viral packaging of the nucleic acid construct with the assistance of a lentivirus packaging plasmid and a cell line. The SRSF1 protein inhibitor is selected from the group consisting of an SRSF1 protein antibody, an SRSF1 protein binding molecule, and an SRSF1 protein degradation agent. As an optional embodiment, the SRSF1 protein inhibitor includes Labetalol and Betaxolol; the Labetalol or the Betaxolol inhibits binding of the SRSF1 protein to downstream RNAs by binding to the SRSF1 protein.

The present disclosure further provides a drug for preventing and/or treating aortic dissection, including a pharmaceutically acceptable carrier and an SRSF1 inhibitor.

In the present disclosure, the pharmaceutically acceptable carrier includes but is not limited to, water, saline, buffer, glycerol, ethanol, liposomes, lipids, proteins, protein-antibody conjugates, peptides, cellulose, nanogels, or a combination thereof. The carrier should be compatible with a dosage form of the drug. The SRSF1 inhibitor is selected from the group consisting of an SRSF1 gene inhibitor and an SRSF1 protein inhibitor. The SRSF1 gene inhibitor is selected from the group consisting of a nucleic acid molecule, a nucleic acid construct, and a lentivirus. The nucleic acid molecule includes SRSF1 gene-specific siRNA, SRSF1 gene-specific shRNA, SRSF1 gene-specific microRNA, and nucleic acid molecules that inhibit SRSF1 gene promoters. As an embodiment, the nucleic acid molecule is a short hairpin RNA (shRNA), and the shRNA includes a sense strand fragment, an antisense strand fragment, and a stem-loop structure (loop sequence) ligating the sense strand fragment and the antisense strand fragment; sequences of the sense strand fragment and the antisense strand fragment are complementary; and the sequence of the sense strand fragment is any one selected from the group consisting of SEQ ID NO: 1, SEQ ID NO: 2, and a sequence complementary thereto. There is no particular limitation on the sequence of the stem-loop structure, which can be routinely selected according to actual demands. The nucleic acid construct carries a gene fragment encoding the nucleic acid molecule and can express the nucleic acid molecule. The lentivirus is formed by viral packaging of the nucleic acid construct with the assistance of a lentivirus packaging plasmid and a cell line. The SRSF1 protein inhibitor is selected from the group consisting of an SRSF1 protein antibody, an SRSF1 protein binding molecule, and an SRSF1 protein degradation agent. As an optional embodiment, the SRSF1 protein inhibitor includes Labetalol and Betaxolol; the Labetalol or the Betaxolol inhibits binding of the SRSF1 protein to downstream protein RNAs by binding to the SRSF1 protein. There is no special limitation on the dosage form of the drug, which may be an injection, an oral preparation (tablet, capsule, and oral solution), a transdermal preparation, and a sustained-release preparation. There is no special limitation on the method of administration, and injection, oral administration, and topical administration can be routinely selected according to the drug dosage form and actual demands.

In the present disclosure, as an embodiment, a method for treating aortic dissection includes: treating the aortic dissection by reducing an expression level of SRSF1 gene or protein. A method for reducing the expression level of SRSF1 gene or protein includes: specifically knocking down SRSF1 by gene knockout technology, and using a drug containing SRSF1 gene or protein inhibitor.

The technical solutions provided by the present disclosure will be described in detail below with reference to examples, but the examples should not be construed as limiting the claimed scope of the present disclosure.

In the following examples, all methods are conventional methods, unless otherwise specified.

The materials, reagents, and the like used in the following examples are all commercially available, unless otherwise specified.

### Example 1

(1) Determination of SRSF1 Expression Level in Patients with Aortic Dissection

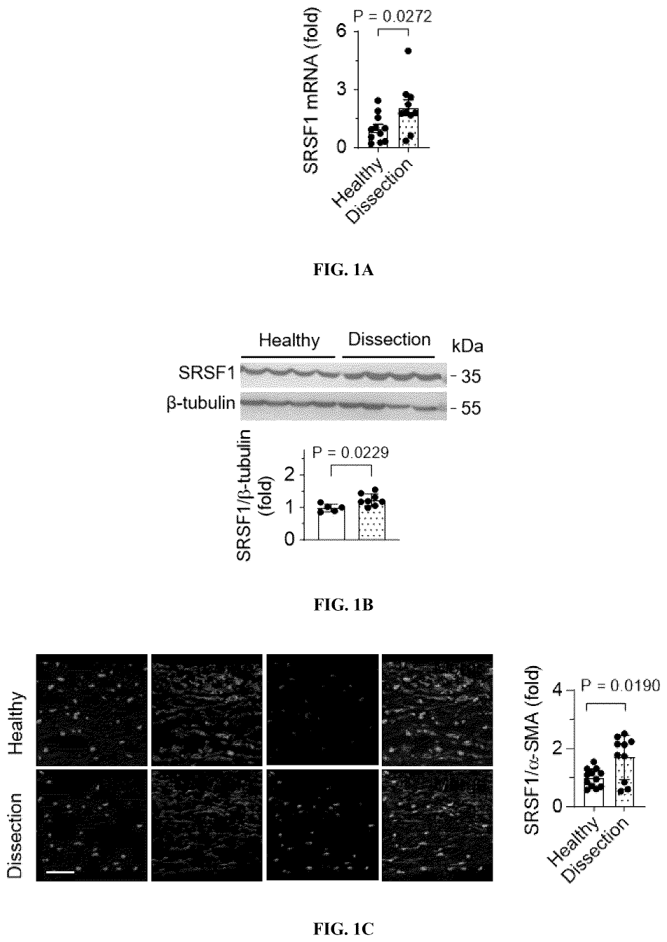

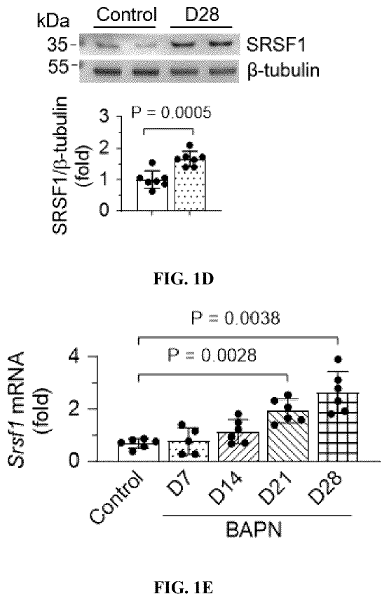

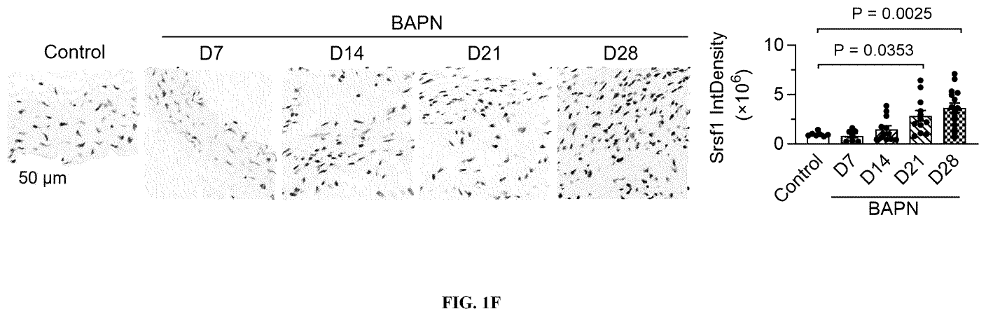

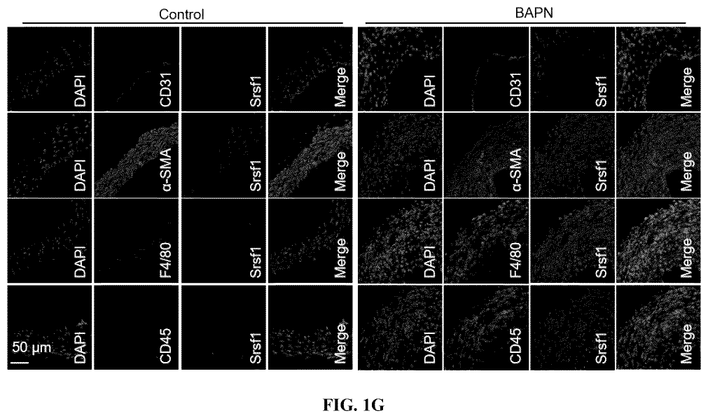

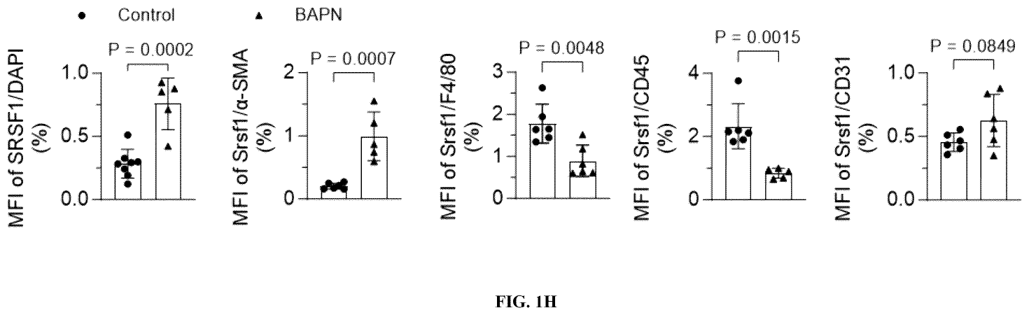

Ten aortic tissues were separately obtained from patients with aortic dissection and from healthy subjects, and the SRSF1 mRNA level and protein level in the aortic tissues were detected, respectively, as shown in FIG. 1A and FIG. 1B. The immunofluorescence staining of SRSF1 in aortic tissues of patients with aortic dissection and healthy subjects was conducted, and the results are shown in FIG. 1C. Compared with those of healthy controls, SRSF1 mRNA and protein levels were upregulated in patients with aortic dissection, and immunofluorescence staining results showed that the fluorescence expression of SRSF1 was upregulated in patients with aortic dissection.

(2) Determination of SRSF1 Expression Level in Mice with Aortic Dissection

Establishment of a mouse model of aortic dissection: Forty 3-week-old wild-type C57 mice were selected, and 0.5% BAPN (i.e., 0.5 g of BAPN added into 100 mL of water) was added to the drinking water, and the mice were fed with BAPN drinking water for 4 weeks. 3-week-old wild-type C57 mice (10 mice) that were fed drinking water without BAPN for 4 weeks were used as a control (Control group).

10 mice were sacrificed on the 7th, 14th, 21st, and 28th day after the start of BAPN drinking water feeding. A whole aorta tissue was obtained, and aortic sections were made after partial fixation. Immunofluorescence staining was conducted to detect the expression of SRSF1 protein. The remaining aortic tissue was snap-frozen in liquid nitrogen, one part of which was used to extract RNA and detect the mRNA expression of SRSF1 by quantitative polymerase chain reaction (QPCR), and the other part of which was used to extract protein and detect the expression of SRSF1 protein by Western Blot. The results are shown in FIG. 1D to FIG. 1H. The mRNA and protein levels of SRSF1 were upregulated during aortic dissection in mice (FIG. 1D to FIG. 1E). Immunohistochemistry and immunofluorescence results showed that the fluorescence expression of SRSF1 was upregulated in ascending aortic dissection (FIG. 1F to FIG. 1H).

### Example 2

(1) Construction of smooth muscle cell-specific SRSF1 knockdown mice Srsf1^{f/f}; SM22^{Cre-ERT2}: Loxp sites were inserted at both ends of SRSF1 exon to obtain loxp-exons-loxp mice (SRSF1^{flox/flox} homozygous mice). The offspring obtained by hybridizing SRSF1^{flox/flox} homozygous mice and SM22-cre^{ERT2} mice were genotyped. Under the induction of tamoxifen, the exon sequence of SRSF1 in smooth muscle cells of Cre-positive mice was deleted, such that SRSF1 protein was knocked out only in smooth muscle cells, and Srsf1^{f/f}; SM22^{Cre-ERT2} mice were obtained.

(2) Experimental grouping: thirty-one 3-week-old Srsf1^{f/f}; SM22^{Cre-ERT2} mice and 34 Srsf1^{f/f} mice from the same litter were selected. At the age of 3 weeks, the mice were simultaneously given BAPN (concentration 0.5%) in drinking water and fed for 4 weeks, and were designated as BAPN+Srsf1^{f/f};SM22^{Cre-ERT2} group and BAPN+Srsf1^{f/f} group. Fifteen Srsf1^{f/f}; SM22^{Cre-ERT2} mice of the same species and age and fifteen Srsf1^{f/f} mice from the same litter were selected. At the age of 3 weeks, the mice were simultaneously given drinking water without BAPN and fed for 4 weeks, and were designated as Srsf1^{f/f}; SM22^{Cre-ERT2} group and Srsf1^{f/f} group.

### (3) Experimental Determination:

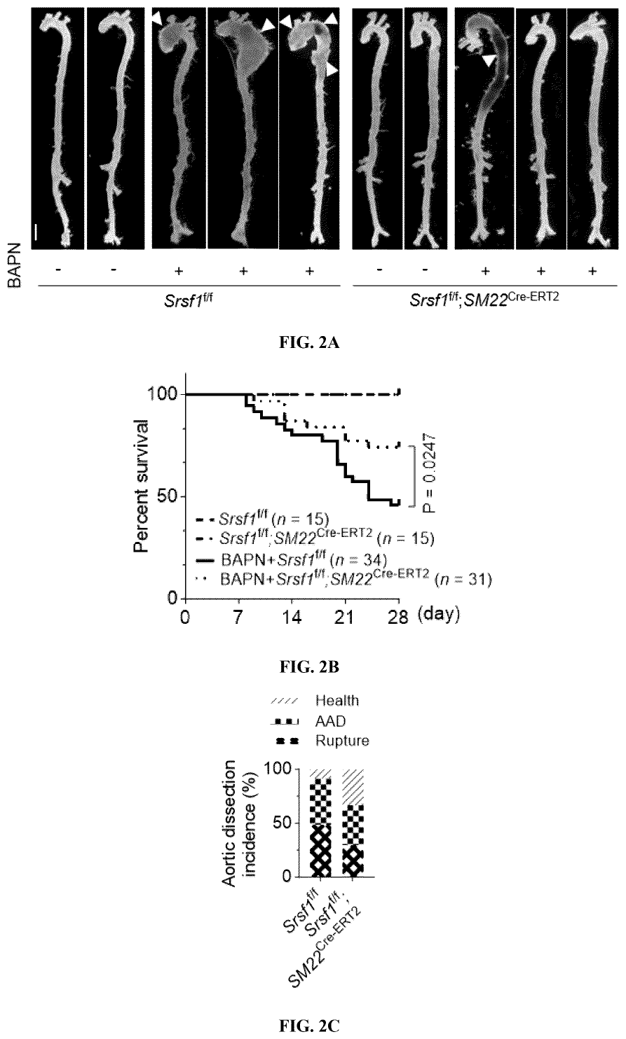

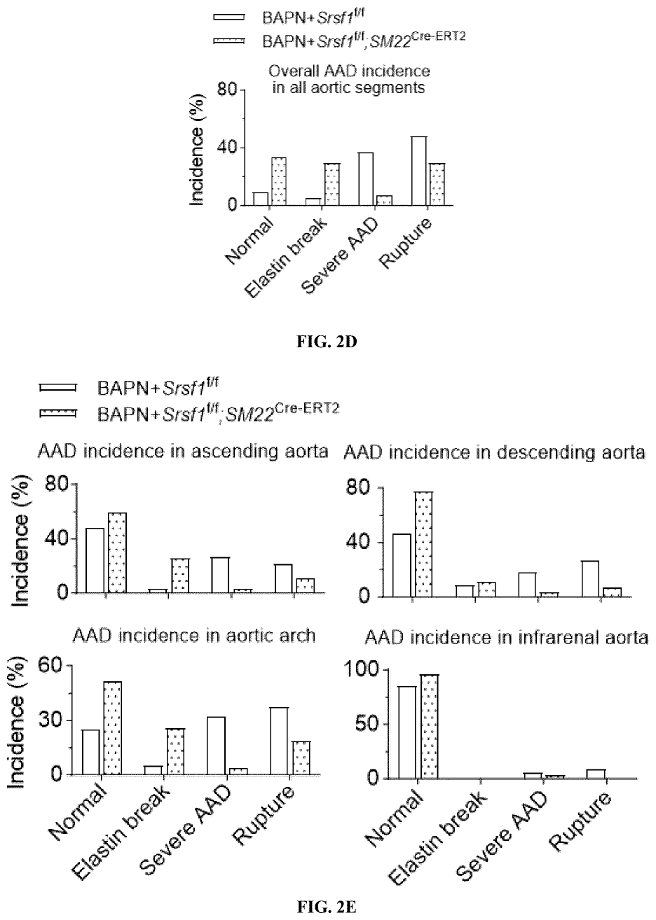

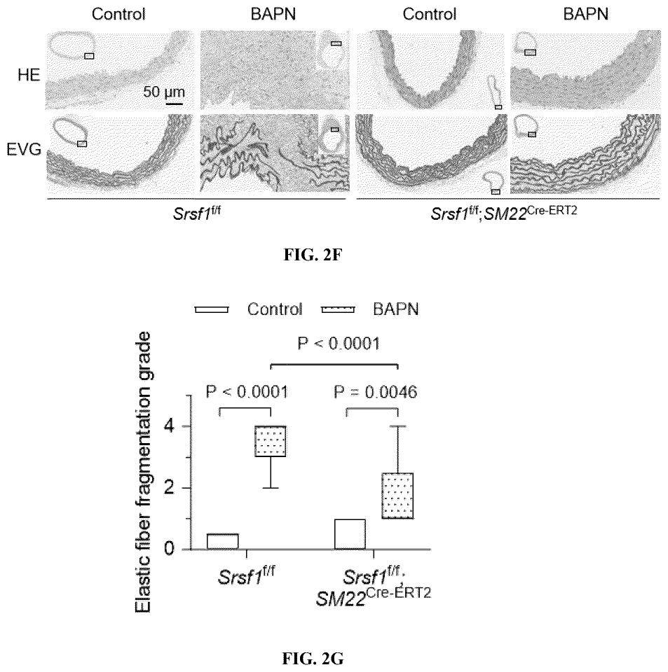

On the 28th day of induction, the whole aorta tissues of the 4 groups of mice were taken and photographed under a stereomicroscope to compare the changes in aortic vascular morphology and the severity of dissection. The results are shown in FIG. 2A. BAPN induction caused congestion in the vascular wall of different locations in the aorta of Srsf1^{f/f} mice, while the smooth muscle cell-specific knockout of SRSF1 in mice Srsf1^{f/f}; SM22^{Cre-ERT2} was able to improve this phenomenon.

The mortality of mice in each group was counted, and the results are shown in FIG. 2B. BAPN-induced knockout mice (BAPN+Srsf1^{f/f};SM22^{Cre-ERT2}) showed reduced mortality compared with BAPN-induced non-knockout mice (BAPN+Srsf1^{f/f}).

Dissection observations were conducted on the 28th day of induction, and the rates of different degrees of aortic dissection in the ascending aorta, descending aorta, aortic arch, abdominal aorta, and all aortic segments of BAPN-induced non-knockout mice and knockout mice were recorded. The results are shown in FIG. 2C to FIG. 2E. Compared with those in non-knockout mice, SRSF1 knockout inhibited the rate of BAPN-induced acute aortic dissection (AAD) and vascular rupture, and was able to inhibit the formation of aortic dissection in mice.

On the 28th day of induction, the ascending aortas of the 4 groups of mice were fixated, embedded in paraffin, and sectioned, and hematoxylin-eosin (HE) and Elastic Van Gieson (EVG) staining were conducted to compare whether there were any differences in vascular elastic fiber rupture. The results are shown in FIG. 2F to FIG. 2G. Compared with those in non-knockout mice, the degradation of extracellular elastic fibers was significantly reduced in BAPN-induced knockout mice (BAPN+Srsf1^{f/f}; SM22^{Cre-ERT2}).

### Example 3

The inflammation levels and extracellular matrix degradation of the 4 groups of mice at day 28 after BAPN induction in Example 2 were measured, respectively.

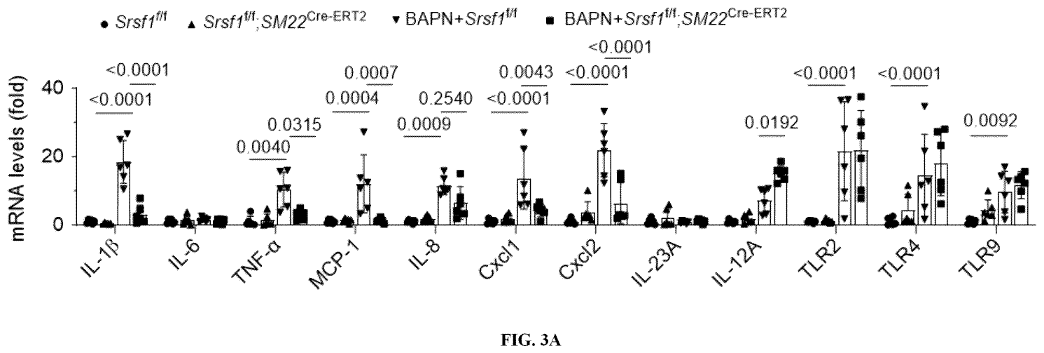

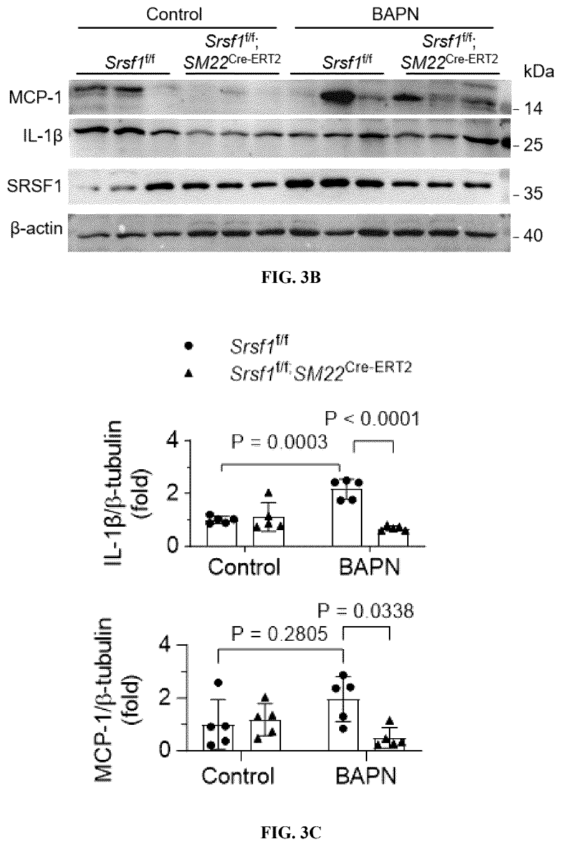

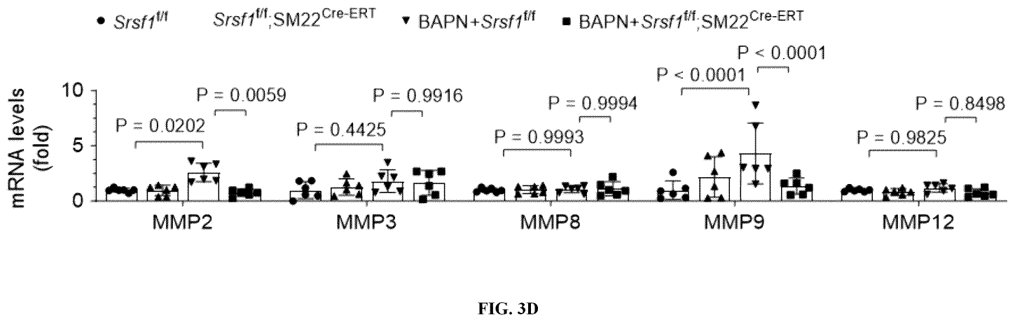

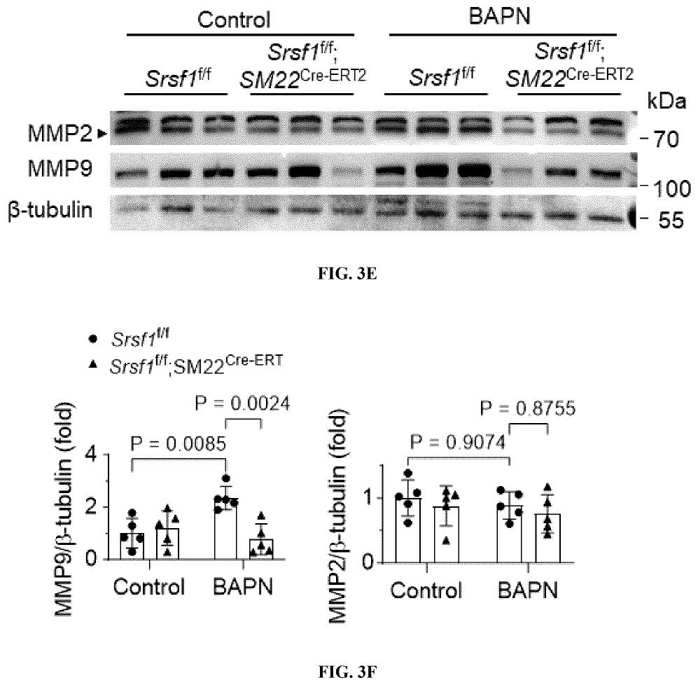

The aortic tissues of each group of mice that were sanp-frozen in liquid nitrogen were taken out and used to detect the mRNA levels and protein levels of inflammatory factors (IL-1β, IL-6, TNF-α, MCP-1, IL-8, Cxcl1, Cxcl2, IL-23A, IL-12A, TLR2, TLR4, and TLR9) and matrix metalloproteinases (MMP2, MMP3, MMP8, MMP9, and MMP12). The results are shown in FIG. 3. BAPN induction could significantly increase the mRNA and protein expression of inflammatory factors, while SRSF1 knockout inhibited the increase in the expression level of inflammatory factors induced by BAPN and reduced the mRNA/protein level of inflammatory factors. BAPN induction could increase the mRNA and protein expression of extracellular matrix metalloproteinases, while SRSF1 knockout inhibited the increase in the expression level of extracellular matrix metalloproteinases induced by BAPN.

### Example 4

(1) Construction of SRSF1 overexpression mice: in order to verify the influence of SRSF1 on aortic dissection in vivo, SRSF1 overexpression mice were constructed. Adeno-associated virus overexpressing SRSF1 (AAV-Srsf1-GFP) was purchased from Vital River. The adeno-associated virus overexpressing SRSF1 (AAV-Srsf1-GFP) was injected into the tail vein of 3-week-old wild-type C57 mice (WT) at an injection volume of 10¹² v.g./mouse to obtain SRSF1-overexpressing mice.

(2) Experimental grouping: thirty mice with SRSF1 overexpression were selected and fed with BAPN (concentration of 0.5%) in drinking water for 4 weeks, which was designated as the BAPN+AAV-Srsf1-GFP group. Additionally, 31 mice of the same species and age were injected with the same dosage of adeno-associated virus (AAV-GFP) without SRSF1 overexpression as a control group. They were then fed with BAPN (concentration of 0.5%) in drinking water for 4 weeks, which was designated as a BAPN+AAV-GFP group.

### (3) Experimental Determination:

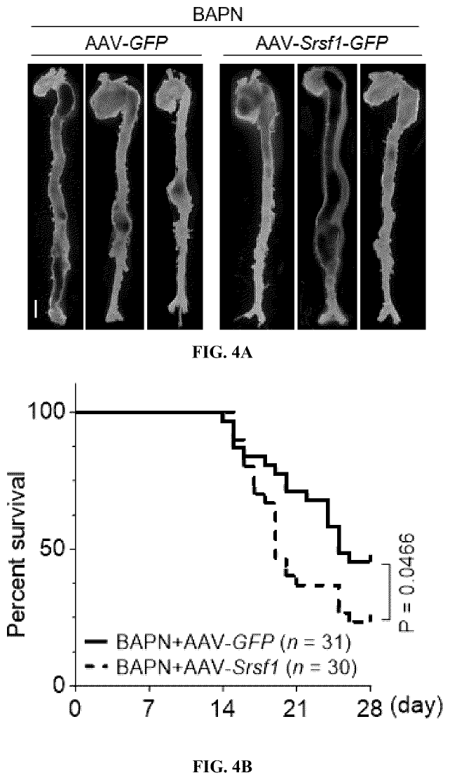

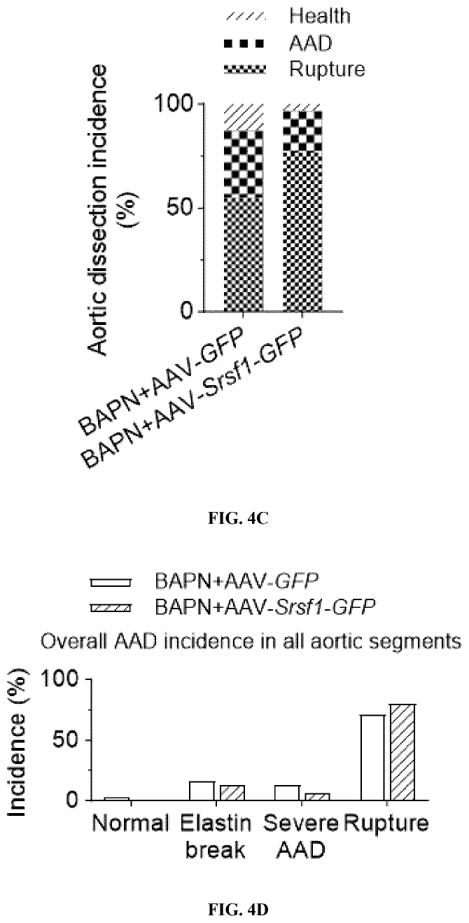

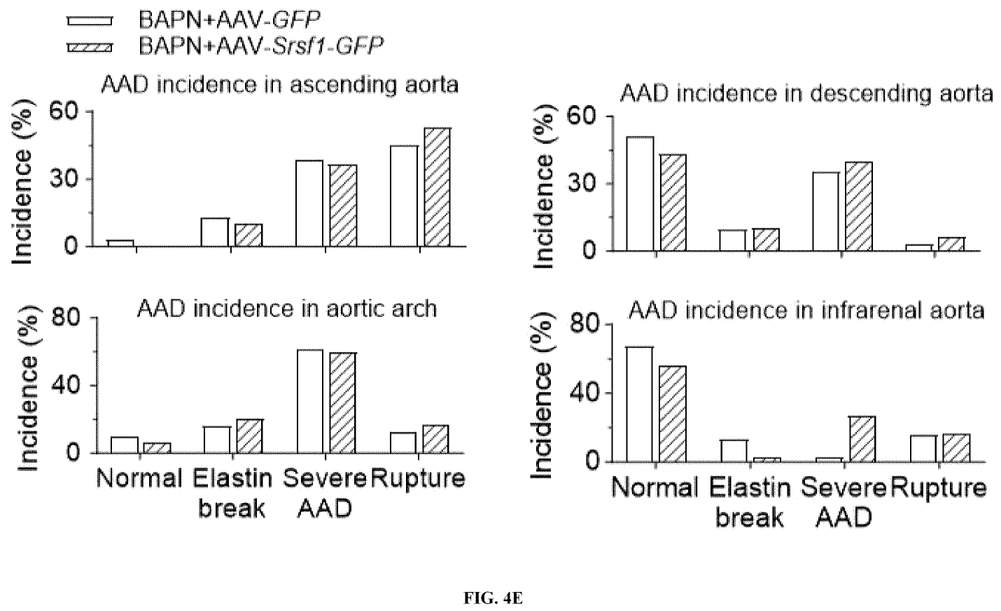

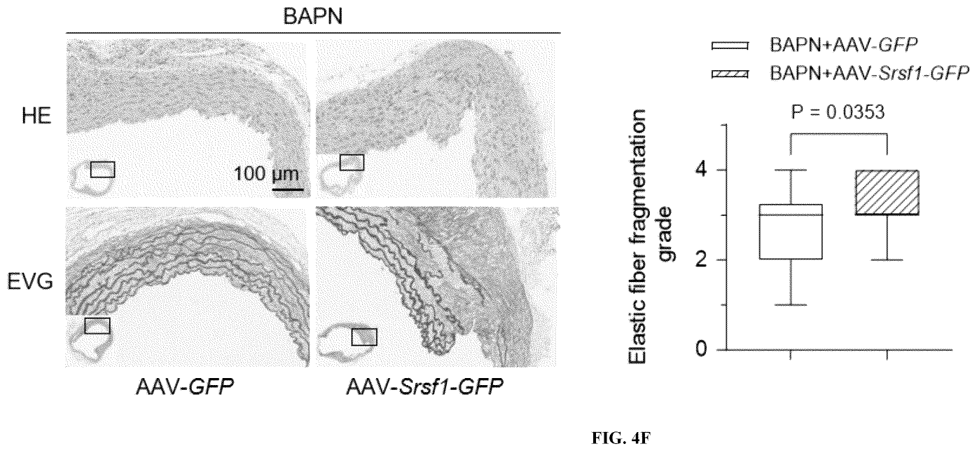

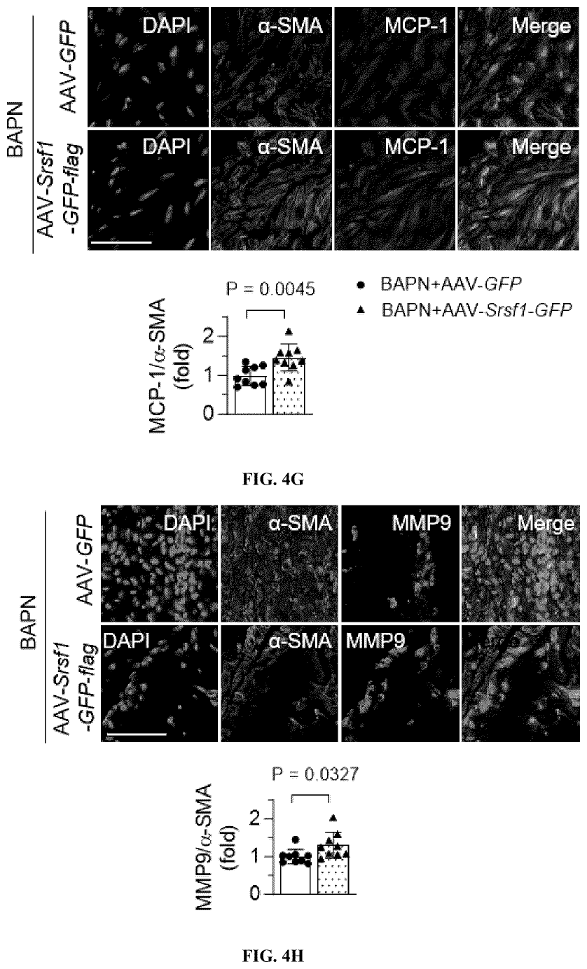

On the 28th day of induction, the whole aorta tissues of the 2 groups of mice were taken and photographed under a stereomicroscope to compare the changes in aortic vascular morphology and the severity of dissection. The results are shown in FIG. 4A. SRSF1-overexpressing mice exhibited more severe aortic dissection.

The mortality of mice in the 2 groups was statistically analyzed, and the results are shown in FIG. 4B. The mortality rate of SRSF1 overexpressing mice was significantly increased.

The mice were sacrificed on the 28th day of induction, and the aorta was dissected and isolated for observation. The formation rates of different degrees of aortic dissection in the ascending aorta, descending aorta, aortic arch, abdominal aorta, and all aortic segments of BAPN-induced overexpression mice (BAPN+AAV-Srsf1-GFP) and control mice (BAPN+AAV-GFP) were recorded. The results are shown in FIG. 4C to FIG. 4E. SRSF1 overexpressing mice showed a more pronounced increase in intercalation.

On the 28th day of induction, the ascending aortas of the 2 groups of mice were fixated, embedded in paraffin, and sectioned. HE and EVG staining were conducted to compare whether there were any differences in vascular elastic fiber rupture. The results are shown in FIG. 4F. The degradation of extracellular elastic fibers was significantly increased in SRSF1-overexpressing mice.

The aortic tissues of the 2 groups of mice were taken out after snap-freezing in liquid nitrogen, and the mRNA and protein levels of inflammatory factors and matrix metalloproteinases were detected by immunofluorescence staining. The results are shown in FIG. 4G to FIG. 4H. SRSF1-overexpressing mice showed increased vascular inflammation and increased expression of extracellular matrix metalloproteinases.

### Example 5

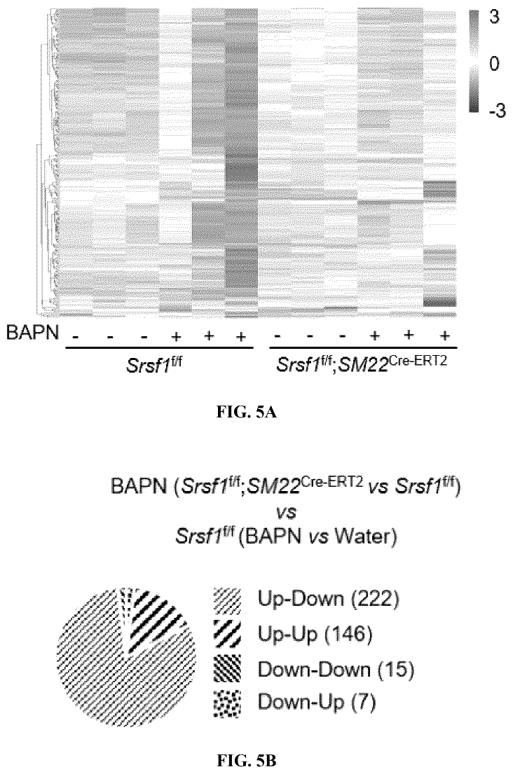

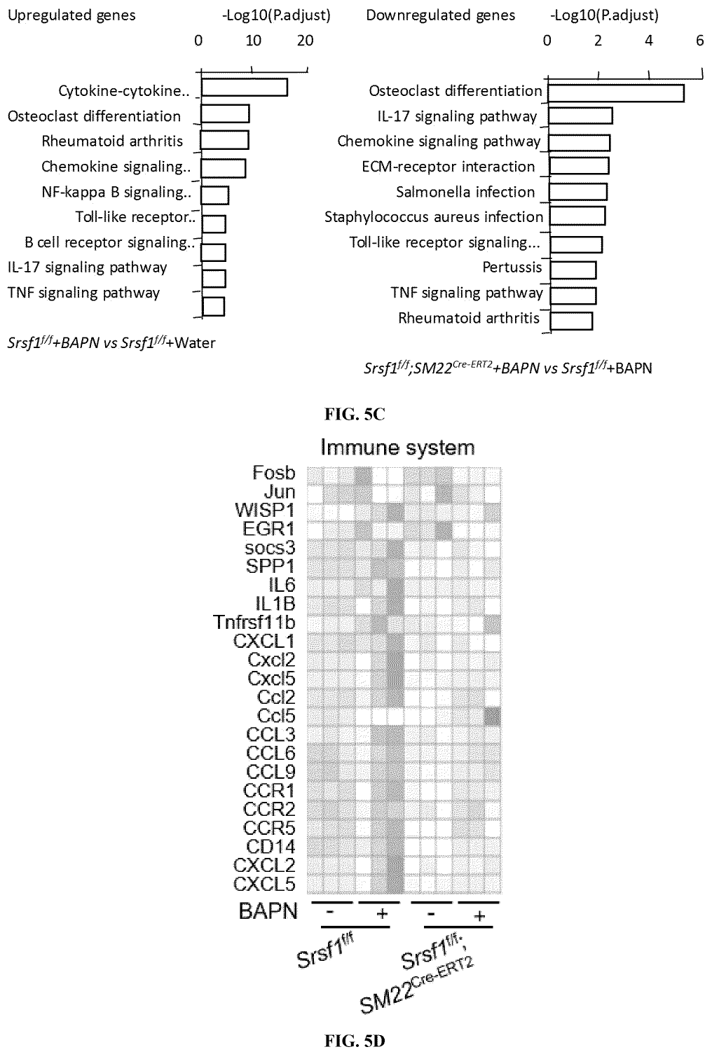

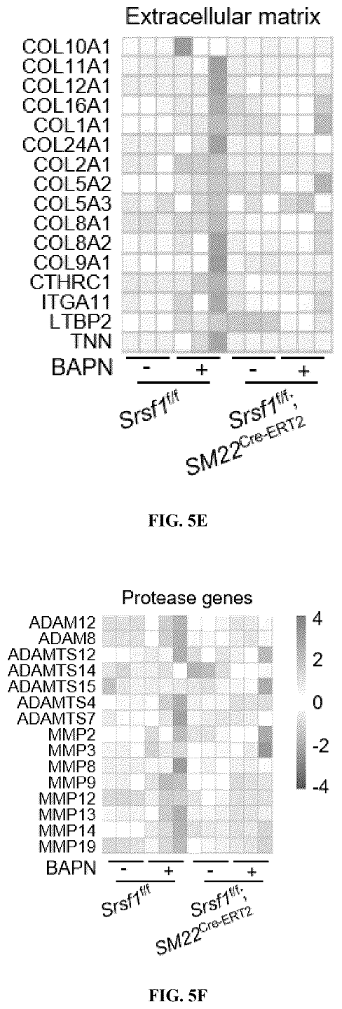

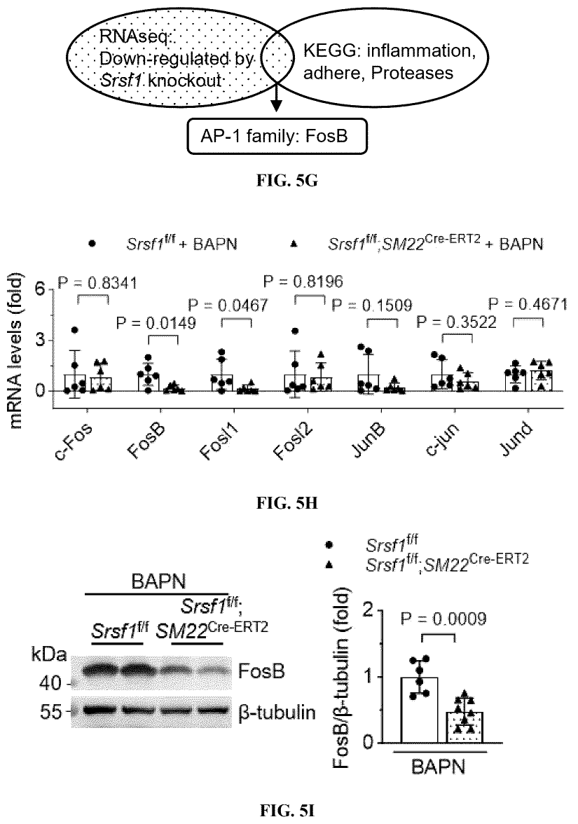

In order to study the mechanism by which SRSF1 regulated the formation of aortic dissection, the 4 groups of experimental mice in Example 2 were taken and the mouse aortic tissues were dissected. RNA was then extracted for high-throughput sequencing. The results are shown in FIG. 5A. The downstream target genes of SRSF1 were searched, and the results are shown in FIG. 5B. There were 222 genes upregulated in Srsf1^{f/f} mice under BAPN treatment compared with water-fed mice, and downregulated in knockout mice and control mice under BAPN treatment. The differentially expressed genes were enriched using a KEGG method, and the results are shown in FIG. 5C. Inflammatory pathways were extensively enriched. Gene expression heat maps of inflammatory factors, extracellular matrix, and matrix metalloproteinases in the 4 groups of mice were plotted, and the results are shown in FIG. 5D to FIG. 5F. Under BAPN treatment, the extracellular matrix proteins, inflammatory factors, and matrix metalloproteinases were downregulated in knockout mice compared with control mice. The expression levels of API family gene mRNA and FosB protein in the aorta tissues of Srsf1^{f/f}; SM22^{Cre-ERT2} mice and control mice after BAPN induction were detected, and the results are shown in FIG. 5H to FIG. 5I. Some genes of the API family were altered in the knockout mice, and FosB mRNA and protein levels were downregulated.

### Example 6

To verify whether SRSF1 regulated the inflammation of smooth muscle cells through FosB, human aortic smooth muscle cells (HASMCs) with knockdown and overexpression of SRSF1 were constructed.

Knockdown of SRSF1 in HASMCs using SRSF1-shRNA: two shRNAs were designed for the coding region of SRSF1 to specifically knockdown the expression level of SRSF1, namely, SRSF1-sh1 and SRSF1-sh2. The sequences were as follows: for SRSF1-sh1, the sense strand fragment was CGGAAAGAAGATATGACGTAT (SEQ ID NO: 1), the antisense strand fragment was ATACGTCATATCTTCTTTCCG (SEQ ID NO: 3), and the stem-loop structure was CTCGAG; for SRSF1-sh2, the sense strand fragment was TATCTGAAGAGATGGATTAAG (SEQ ID NO: 2), the antisense strand fragment was CTTAATCCATCTCTTCAGATA (SEQ ID NO: 4), and a stem-loop structure was CTCGAG. The two shRNAs were ligated to a pLKO.1 vector to obtain SRSF1 gene interference vectors pLKO-SRSF-sh1 and pLKO-SRSF-sh2. The gene interference vectors were transfected into 293 cells together with a packaging plasmid (psPAX2 lentivirus packaging plasmid (with plasmid pMD2G)). After 48 h, the medium was collected to obtain sh1RNA virus and sh2RNA virus. The sh1RNA virus and sh2RNA virus were added into HASMCs. After 48 h, the medium was changed to obtain HASMCs with SRSF1 knockdown, which were referred to as SRSF1.sh1 and SRSF1.sh2. Normal HASMCs (NT) were used as a control.

Overexpression of SRSF1 in HASMCs using Ad-SRSF1: The cultured smooth muscle cells treated with Ad-SRSF1 at a titer of 100 MOI were designated as Ad-SRSF1. The cultured smooth muscle cells treated with Ad-GEP at the same titer were set as a control, and designated as Ad-GEP.

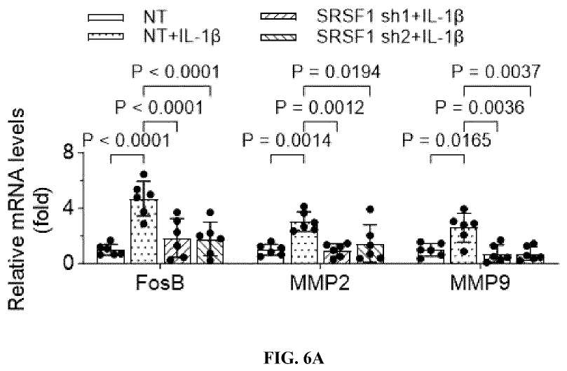

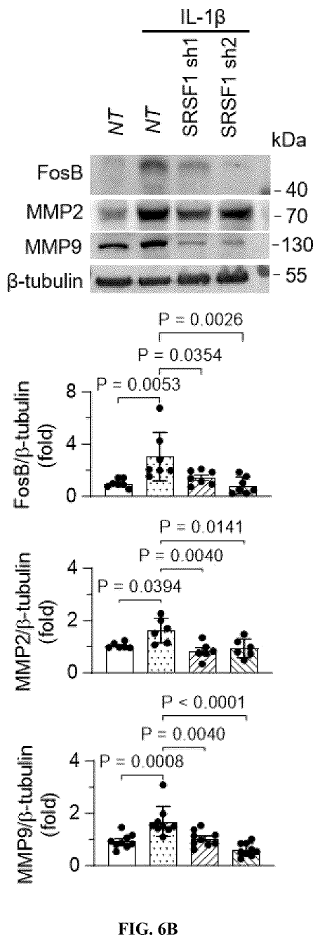

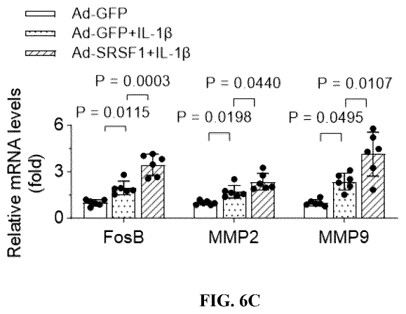

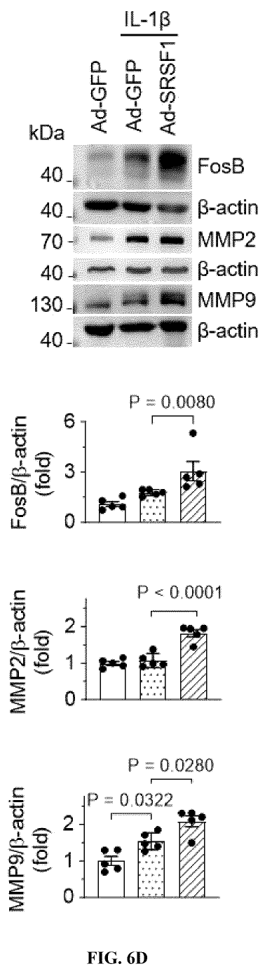

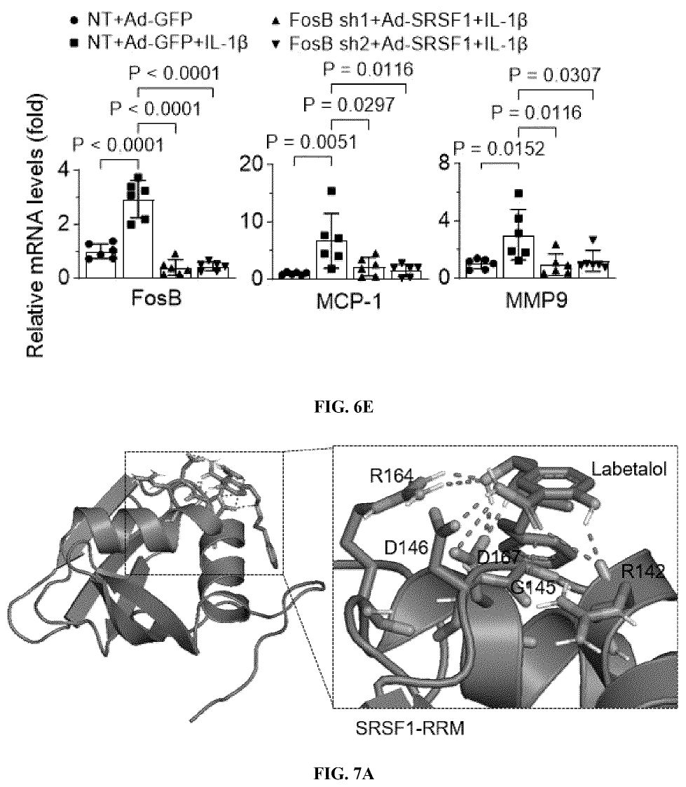

IL-1β was added to the medium of NT, SRSF1 sh1, SRSF1 sh2, Ad-SRSF1, and Ad-GEP cells to simulate aortic dissection in the cells, while NT and Ad-GEP without IL-1β were used as controls. The mRNA and protein expressions of FosB and MMP2/MMP9 in each group of cells were detected, and the results are shown in FIG. 6A to FIG. 6D. The addition of IL-1β upregulated the mRNA and protein expression levels of FosB, MMP2, and MMP9; SRSF1 knockdown could inhibit the upregulation of FosB, MMP2, and MMP9 expression (FIG. 6A and FIG. 6B), while overexpression of SRSF1 could further promote the upregulation of FosB, MMP2, and MMP9 expression (FIG. 6C and FIG. 6D).

Knockdown of FosB in HASMCs overexpressing SRSF1 (Ad-SRSF1) using shRNA: two shRNAs were designed to target the coding region of FosB to specifically knockdown the expression level of FosB in Ad-SRSF1 cells, namely, FosB-sh1 and FosB-sh2. The sequences were as follows: for FosB-sh1, the sense strand fragment was AGGTCACGTTGGCCCTCAA (SEQ ID NO: 5), the antisense strand fragment was TTGAGGGCCAACGTGACCT (SEQ ID NO: 6), and the stem-loop structure was CTCGAG; and for FosB-sh2, the sense strand fragment was GAGGAAGAGGAGAAGCGAA (SEQ ID NO: 7), the antisense strand fragment was TTCGCTTCTCCTCTTCCTC (SEQ ID NO: 8), and the stem-loop structure was CTCGAG. HASMCs overexpressing SRSF1 with knockdown of FosB were constructed, and designated as FosB sh1+Ad-SRSF1 and FosB sh2+Ad-SRSF1. Ad-GFP without knockdown of FosB was used as a control, and designated as NT+Ad-GFP.

IL-1β was added to the medium of NT+Ad-GFP, FosB sh1+Ad-SRSF1, and FosB sh2+Ad-SRSF1 cells, while NT+Ad-GFP without IL-1β was used as a control. The influence of overexpressing SRSF1 after knocking down FosB on the expression of FosB and matrix metalloproteinases was detected, and the results are shown in FIG. 6E. Knockdown of FosB inhibited the expression of MMPs induced by SRSF1.

It was concluded that SRSF1 promoted inflammation in smooth muscle cells through FosB.

### Example 7

(1) Experimental grouping: Fifteen 3-week-old wild-type C57 mice were intraperitoneally injected with 5 mg/kg/d of SRSF1 inhibitor Labetalol (MCE, HY-121383) and fed with drinking water for 4 weeks, designating as Labetalol group. Fifteen mice of the same species and age were given the same dosage of saline in the same way as a control group and fed with drinking water for 4 weeks, designating as Saline group. Thirty mice of the same species and age were given Labetalol in the same way and at the same dosage, and fed with BAPN (concentration of 0.5%) through drinking water for 4 weeks, designating as BAPN+Labetalol group. Twenty-nine mice of the same species and age were given saline in the same way and at the same dosage, and fed with BAPN (concentration of 0.5%) through drinking water for 4 weeks, designating as a BAPN+saline group.

### (2) Experimental Determination:

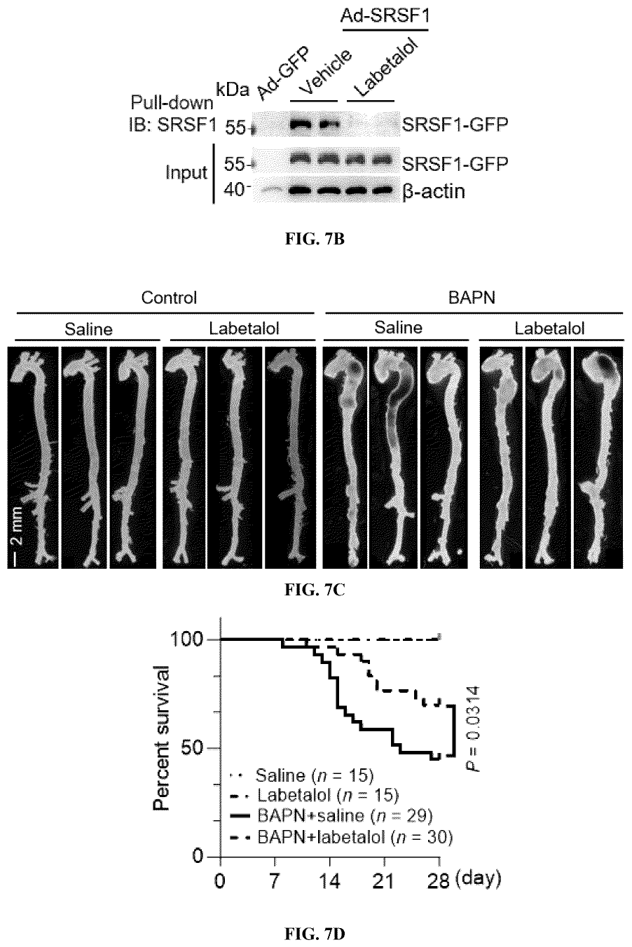

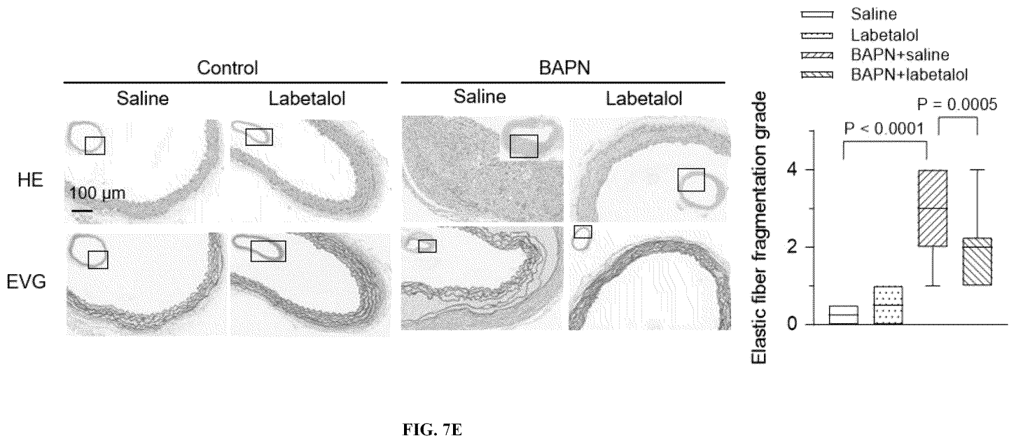

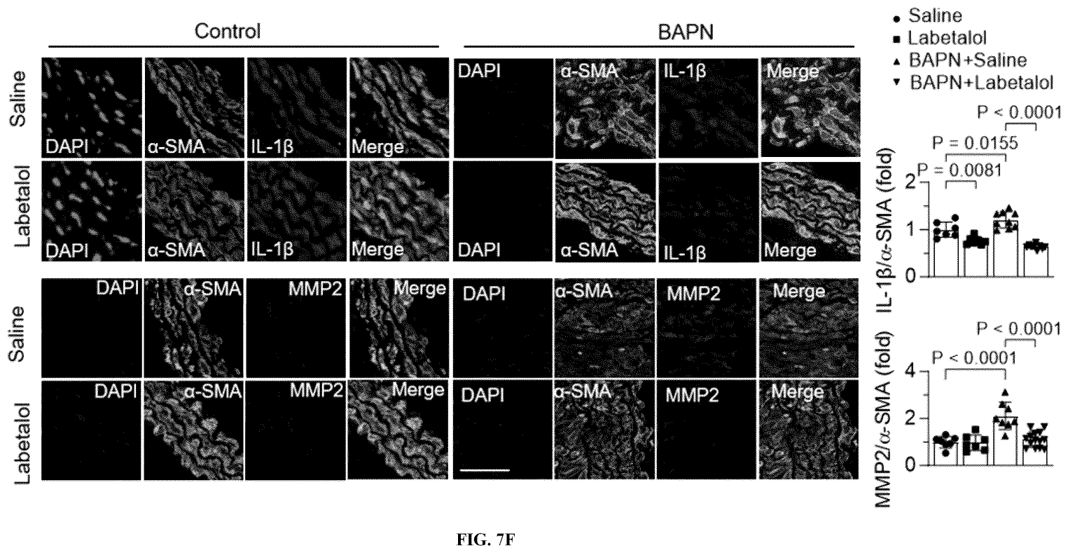

The binding mode of Labetalol and SRSF1 was obtained by molecular docking, and the results are shown in FIG. 7A. The amino acid residues in the RRM region of SRSF1 was covalently bound to Labetalol.

SRSF1 was overexpressed in smooth muscle cells in the same manner as that in Example 6 to obtain Ad-SRSF1, while Ad-GFP was used as a control. 10 μmol of Labetalol was added to the Ad-SRSF1 medium, while a control without Labetalol was used (Vehicle). The binding of SRSF1 and RNA was detected by RNA pull down method. The results are shown in FIG. 7B, which indicates that Labetalol could inhibit the binding of SRSF1 and downstream RNA.

On the 28th day of induction, the whole aorta tissues of the 4 groups of mice were taken and photographed under a stereomicroscope to compare the changes in aortic vascular morphology and the severity of dissection. The results are shown in FIG. 7C. Aortic dissection was reduced in mice injected with SRSF1 inhibitors under BAPN induction.

The mortality of the 4 groups of mice was statistically analyzed, and the results are shown in FIG. 7D. It is indicated that the mortality rate of mice injected with SRSF1 inhibitor under BAPN induction was lower than that of control mice.

On the 28th day of induction, the ascending aortas of the 4 groups of mice were fixated, embedded in paraffin, and sectioned. HE and EVG staining were conducted to compare whether there were any differences in vascular elastic fiber rupture. The results are shown in FIG. 7E. Under BAPN induction, extracellular elastic fiber degradation was reduced in mice injected with SRSF1 inhibitor compared with that in control mice.

On the 28th day of induction, the mRNA levels and protein levels of inflammatory factors and extracellular matrix proteases in the aorta tissues of the 4 groups of mice were measured, and the results are shown in FIG. 7F. Under BAPN induction, the mRNA and protein levels of inflammatory factors and extracellular matrix proteases in mice injected with SRSF1 inhibitor were reduced compared with those in control mice.

The above descriptions are merely preferred embodiments of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A method for screening a drug for prevention and/or treatment of aortic dissection, comprising using a serine/arginine-rich splicing factor 1 (SRSF1) as a target.

2. The method according to claim 1, wherein the SRSF1 is selected from the group consisting of an SRSF1 gene and a SRSF1 protein.

3. The method according to claim 1, wherein overexpression of the SRSF1 promotes occurrence of the aortic dissection, and low expression of the SRSF1 inhibits the occurrence of the aortic dissection.

4. A method for preventing and/or treating aortic dissection, comprising administering a therapeutically effective amount of an SRSF1 inhibitor to a subject in need thereof.

5. The method according to claim 4, wherein the inhibitor is selected from the group consisting of an SRSF1 gene inhibitor and an SRSF1 protein inhibitor.

6. The method according to claim 5, wherein the SRSF1 gene inhibitor is selected from the group consisting of a nucleic acid molecule, a nucleic acid construct, and a lentivirus.

7. The method according to claim 6, wherein the nucleic acid molecule is a short hairpin RNA (shRNA), and the shRNA comprises a sense strand fragment, an antisense strand fragment, and a stem-loop structure ligating the sense strand fragment and the antisense strand fragment; sequences of the sense strand fragment and the antisense strand fragment are complementary; and the sequence of the sense strand fragment is any one selected from the group consisting of SEQ ID NO: 1, SEQ ID NO: 2, and a sequence complementary thereto.

8. The method according to claim 5, wherein the SRSF1 protein inhibitor is selected from the group consisting of an SRSF1 protein antibody, an SRSF1 protein binding molecule, and an SRSF1 protein degradation agent.

9. A drug for preventing and/or treating aortic dissection, comprising a pharmaceutically acceptable carrier and an SRSF1 inhibitor.

10. The drug according to claim 9, wherein the inhibitor is selected from the group consisting of an SRSF1 gene inhibitor and an SRSF1 protein inhibitor.


SEQUENCE LISTING (8 sequences)

SEQ ID NO: 1 - RNA, 21 nt, synthetic construct
  source 1..21: mol_type = other RNA; note = Sense strand fragment of SRSF1-sh1
cggaaagaagatatgacgtat

SEQ ID NO: 2 - RNA, 21 nt, synthetic construct
  source 1..21: mol_type = other RNA; note = Sense strand fragment of SRSF1-sh2
tatctgaagagatggattaag

SEQ ID NO: 3 - RNA, 21 nt, synthetic construct
  source 1..21: mol_type = other RNA; note = Anti-Sense strand fragment of SRSF1-sh1
atacgtcatatcttctttccg

SEQ ID NO: 4 - RNA, 21 nt, synthetic construct
  source 1..21: mol_type = other RNA; note = Anti-Sense strand fragment of SRSF1-sh2
cttaatccatctcttcagata

SEQ ID NO: 5 - RNA, 19 nt, synthetic construct
  source 1..19: mol_type = other RNA; note = Sense strand fragment of FosB-sh1
aggtcacgttggccctcaa

SEQ ID NO: 6 - RNA, 19 nt, synthetic construct
  source 1..19: mol_type = other RNA; note = Anti-sense strand fragment of FosB-sh1
ttgagggccaacgtgacct

SEQ ID NO: 7 - RNA, 19 nt, synthetic construct
  source 1..19: mol_type = other RNA; note = Sense strand fragment of FosB-sh2
gaggaagaggagaagcgaa

SEQ ID NO: 8 - RNA, 19 nt, synthetic construct
  source 1..19: mol_type = other RNA; note = Anti-sense strand fragment of FosB-sh2
ttcgcttctcctcttcctc